(19) United States
(12) Patent Application Publication
(10) Pub. No.: US 2005/0090439 A1
(43) Pub. Date: Apr. 28, 2005

(54) Vaccine for the prevention and treatment of Alzheimer's and amyloid related diseases

(76) Inventors: Robert Chalifour, Ile Bizard (CA); Lise Hebert, Montreal Nord (CA); Xianqi Kong, Dollard-des-Ormeaux (CA); Francine Gervais, Ile Bizard (CA)

Correspondence Address: CLARK & ELBING LLP, 101 FEDERAL STREET, BOSTON, MA, 02110, US

(21) Appl. No.: 10/825,958

(22) Filed: Apr. 16, 2004

Related U.S. Application Data
(63) Continuation of application No. 09/724,842, filed on Nov. 28, 2000, now abandoned.
(60) Provisional application No. 60/168,594, filed on Nov. 29, 1999.

(51) Int. Cl. A61K 38/16

(52) U.S. Cl. 514/12

(57) ABSTRACT

The present invention relates to a stereochemically based “non-self” antigen vaccine for the prevention and/or treatment of Alzheimer's and other amyloid related diseases. The present invention provides a vaccine for the prevention and treatment of Alzheimer's and other amyloid related diseases, which overcomes the drawbacks associated with using naturally occurring peptides, proteins or immunogens.

### RELATED APPLICATIONS

This application is a continuation of U.S. application Ser. No. 09/724,842, filed Nov. 28, 2000, which claims the benefit of priority under 35 U.S.C. 119(e) to copending U.S. Provisional Application No. 60/168,594, filed on Nov. 29, 1999, the entire contents of each of which are incorporated herein by reference.

### BACKGROUND OF THE INVENTION

The present invention relates to a new stereochemically based “non-self” antigen vaccine for the prevention and/or treatment of Alzheimer's and other amyloid related diseases.

Amyloidosis refers to a pathological condition characterized by the presence of amyloid fibers. Amyloid is a generic term referring to a group of diverse but specific protein deposits (intracellular and/or extracellular) that are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common x-ray diffraction and infrared spectra.

Amyloid-related diseases can either be restricted to one organ or spread to several organs. The first instance is referred to as “localized amyloidosis” while the second is referred to as “systemic amyloidosis.”

Some amyloidotic diseases can be idiopathic, but most of these diseases appear as a complication of a previously existing disorder. For example, primary amyloidosis can appear without any other pathology or can follow plasma cell dyscrasia or multiple myeloma. Secondary amyloidosis is usually seen associated with chronic infection (such as tuberculosis) or chronic inflammation (such as rheumatoid arthritis). A familial form of secondary amyloidosis is also seen in Familial Mediterranean Fever (FMF). This familial type of amyloidosis, as one of the other types of familial amyloidosis, is genetically inherited and is found in specific population groups. In these two types of amyloidosis, deposits are found in several organs and are thus considered systemic amyloid diseases. Another type of systemic amyloidosis is found in long-term hemodialysis patients. In each of these cases, a different amyloidogenic protein is involved in amyloid deposition.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the plaque and blood vessel amyloid is formed by the deposition of fibrillar Aβ amyloid protein. Other diseases such as adult-onset diabetes (Type II diabetes) are characterized by the localized accumulation of amyloid in the pancreas.

Once these amyloids have formed, there is no known, widely accepted therapy or treatment which significantly dissolves the deposits in situ.

Each amyloidogenic protein has the ability to organize into β-sheets and to form insoluble fibrils which get deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences which, although different, will show similarities such as regions with the ability to bind to the glycosaminoglycan (GAG) portion of proteoglycan (referred to as the GAG binding site) as well as other regions which will promote β-sheet formation.

In specific cases, amyloidotic fibrils, once deposited, can become toxic to the surrounding cells. As per example, the Aβ fibrils organized as senile plaques have been shown to be associated with dead neuronal cells and microgliosis in patients with Alzheimer's disease. When tested in vitro, Aβ peptide was shown to be capable of triggering an activation process of microglia (brain macrophages), which would explain the presence of microgliosis and brain inflammation found in the brain of patients with Alzheimer's disease.

In another type of amyloidosis seen in patients with Type II diabetes, the amyloidogenic protein IAPP has been shown to induce β-islet cell toxicity in vitro. Hence, appearance of IAPP fibrils in the pancreas of Type II diabetic patients could contribute to the loss of the β islet cells (Langerhans) and organ dysfunction.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). A main constituent of these amyloid plaques is the amyloid-β peptide (Aβ), a 40-42 amino-acid protein that is produced through cleavage of the β-amyloid precursor protein (APP). Although symptomatic treatments exist for Alzheimer's disease, this disease cannot be prevented nor cured at this time.

The use of a vaccine to treat Alzheimer's disease is possible in principle (Schenk, D. et al., (1999) Nature 400, 173-177). Schenk et al. show that, in a transgenic mouse model of brain amyloidosis (as seen in Alzheimer's disease), immunization with Aβ peptide inhibits the formation of amyloid plaques and the associated dystrophic neurites. In that study, a vaccine using the human aggregated all-L peptide as immunogen prevented the formation of β-amyloid plaque, astrogliosis and neuritic dystrophy in vaccinated transgenic mice.

However, it is apparent that there are a number of drawbacks to using an endogenous protein as a vaccine (or a protein naturally present in the animal being vaccinated). Some of these drawbacks include:
Possible development of autoimmune disease due to the generation of antibodies against “self” protein.

Difficulty in eliciting an immune response due to the failure of the host immune system to recognize “self” antigens.

Possible development of an acute inflammatory response.

### SUMMARY OF THE INVENTION

The present invention relates to a stereochemically based “non-self” antigen vaccine for the prevention and/or treatment of Alzheimer's and other amyloid related diseases. One aim of the present invention is to provide a vaccine for the prevention and treatment of Alzheimer's and other amyloid related diseases, which overcomes the drawbacks associated with using naturally occurring peptides, proteins or immunogens.

In an embodiment, a vaccine is provided which is produced using a “non-self” peptide or protein synthesized from the unnatural D-configuration amino acids, to avoid the drawbacks of using “self” proteins. In accordance with the present invention, the peptides need not be aggregated to be operative or immunogenic as opposed to the prior art vaccines.

In another embodiment, there is provided a method for preventing and/or treating an amyloid-related disease in a subject, which features administering to the subject an antigenic amount of an all-D peptide which elicits production of antibodies against the all-D peptide, and elicit an immune response by the subject, therefore preventing fibrillogenesis and associated cellular toxicity, wherein the antibodies interact with at least one region of an amyloid protein, e.g., β sheet region and GAG-binding site region, immunogenic fragments thereof, protein conjugates thereof, immunogenic derivative peptides thereof, immunogenic peptides thereof, and immunogenic peptidomimetics thereof. These vaccines may be used in the prevention and/or treatment of amyloid related diseases, and in the manufacture of medicaments for preventing and/or treating amyloid-related diseases.

In a further embodiment of the invention, a vaccine for preventing and/or treating an amyloid-related disease in a subject comprises an antibody which interacts with amyloid proteins to prevent fibrillogenesis, wherein the antibodies are raised against an antigenic amount of an all-D peptide interacting with at least one region of an amyloid protein, e.g., β sheet region and GAG-binding site region, Aβ (1-42, all-D), immunogenic fragments thereof, protein conjugates thereof, immunogenic derivative peptides thereof, immunogenic peptides thereof, and immunogenic peptidomimetics thereof. These vaccines may be used in the prevention and/or treatment of amyloid related diseases, and in the manufacture of medicaments for preventing and/or treating amyloid-related diseases.

Still in a further embodiment, there is provided a method for preventing and/or treating an amyloid-related disease in a subject, which comprises administering to the subject an antigenic amount of an all-D peptide which interacts with at least one region of an amyloid protein, e.g., β sheet region and GAG-binding site region, Aβ (1-42), immunogenic fragments thereof, protein conjugates thereof, immunogenic derivative peptides thereof, immunogenic peptides thereof, and immunogenic peptidomimetics thereof, wherein the compound elicits an immune response by the subject and therefore prevents fibrillogenesis.

In a preferred embodiment of the present invention, the compound is a compound of Formula I:
R′—(P)—R″ (I),
wherein
P is an all-D peptide interacting with at least one region of an amyloid protein, e.g., β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent, e.g.:
   hydrogen;
   lower alkyl groups, e.g., acyclic or cyclic having 1 to 8 carbon atoms, without or with functional groups, e.g., carboxylate, sulfonate and phosphonate;
   aromatic groups;
   heterocyclic groups; and
   acyl groups, e.g., alkylcarbonyl, arylcarbonyl, sulfonyl and phosphonyl groups; and
R″ is a C-terminal substituent, e.g., hydroxy, alkoxy, aryloxy, unsubstituted or substituted amino groups.

In an embodiment, R′ and R″ are identical or different, wherein alkyl or aryl group of R′ and R″ are further substituted with functionalities such as halide (e.g., F, Cl, Br, and I), hydroxyl, alkoxyl, aryloxyl, hydroxycarbonyl, alkoxylcarbonyl, aryloxycarbonyl, carbamyl, unsubstituted or substituted amino, sulfo or alkyloxysulfonyl, phosphono or alkoxyphosphonyl groups.

When the compound has an acid functional group, it can be in the form of a pharmaceutically acceptable salt or ester. When the compound has a basic functional group, it can be in the form of a pharmaceutically acceptable salt.

In a preferred embodiment of the present invention, the subject is a human being.

In yet another embodiment of the present invention, the amyloid related disease may be Alzheimer's disease.

In another embodiment of the present invention, there is provided a method for preventing and/or treating of an amyloid related disease in a subject, comprising administering to the subject an antigenic amount of a compound of Formula I:
R′—(P)—R″ (I),
wherein
P is an all-D peptide interacting with at least one region of an amyloid protein, e.g., β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent selected from the group consisting of:
   hydrogen;
   lower alkyl groups, e.g., acyclic or cyclic having 1 to 8 carbon atoms, without or with functional groups, e.g., carboxylate, sulfonate and phosphonate;
   aromatic groups;
   heterocyclic groups; and
   acyl groups, e.g., alkylcarbonyl, arylcarbonyl, sulfonyl and phosphonyl groups; and
R″ is a C-terminal substituent, e.g., hydroxy, alkoxy, aryloxy, unsubstituted or substituted amino groups.

In accordance with this method, the compound elicits an immune response by the subject, preventing fibrillogenesis.

In accordance with a preferred embodiment of the present invention, there is provided a vaccine for preventing and/or treating an amyloid-related disease in a subject, comprising an antigenic amount of an all-D peptide which interacts with at least one region of an amyloid protein, e.g., β sheet region and GAG-binding site region, Aβ (1-42, all-D) peptide, immunogenic fragments thereof, protein conjugates thereof, immunogenic derivative peptides thereof, immunogenic peptides thereof, and immunogenic peptidomimetics thereof, wherein the compound elicits an immune response by the subject and prevents fibrillogenesis.

### BRIEF DESCRIPTION OF THE DRAWING

FIG. 1 illustrates the targeted sites for the antigenic fragments;

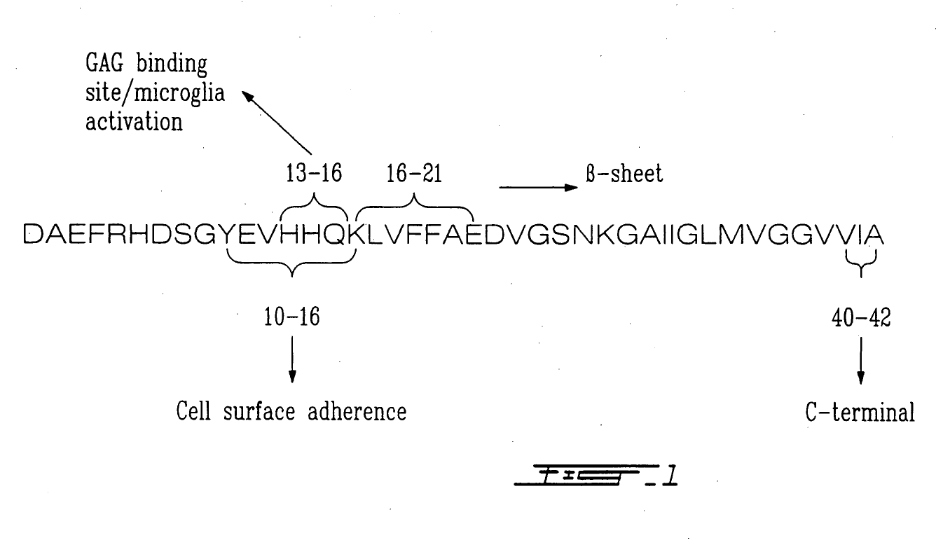

FIG. 2 illustrates the effect of 1 mg/ml of antibodies raised against D and L forms of Aβ(16-21) on fibrillogenesis;

FIG. 3 illustrates the effect of 0.5 mg/ml of antibodies raised against D and L forms of Aβ(16-21) on fibrillogenesis;

FIGS. 4A to 4C illustrate electron micrographs showing the effect of anti-D KLVFFA peptide antibodies (FIG. 4B) and anti-L KLVFFA peptide antibodies (FIG. 4C) with respect to a control (FIG. 4A) on fibrillogenesis;

FIGS. 5A to 5D illustrate the immunohistochemistry of anti-D KLVFFA on aggregated Aβ peptide in brain sections of retrosplenial cortex (FIG. 5A) and parietal cortex (FIG. 5C) and the histochemistry (Thioflavin S assay) of anti-D KLVFFA on aggregated Aβ peptide in the same brain sections of retrospinal cortex (FIG. 5B) and parietal cortex (FIG. 5D);

FIGS. 6A to 6D illustrate the immunohistochemistry of anti-L KLVFFA antibodies on aggregated Aβ peptide in brain sections of parietal cortex (FIG. 6A) and entorhinal cortex (FIG. 6C) and the histochemistry (Thioflavin S assay) of anti-L KLVFFA antibodies on aggregated Aβ peptide in the same brain sections of parietal cortex (FIG. 6B) and entorhinal cortex (FIG. 6D); and

FIG. 7 illustrates the response of rabbits to KLH-conjugated all-L and all-D KLVFFA.

### DETAILED DESCRIPTION OF THE INVENTION

For the purpose of the present disclosure, the following terms are defined below.

The term “peptidomimetic” includes non-peptide compounds which mimic the structural or the functional properties of a peptide.

The term “antigenic fragment thereof” includes fragments of peptides which are capable of eliciting an immune response in a subject.

The term “amyloid related diseases” includes diseases associated with the accumulation of amyloid which can either be restricted to one organ, “localized amyloidosis”, or spread to several organs, “systemic amyloidosis”. Secondary amyloidosis may be associated with chronic infection (such as tuberculosis) or chronic inflammation (such as rheumatoid arthritis), including a familial form of secondary amyloidosis which is also seen in Familial Mediterranean Fever (FMF) and another type of systemic amyloidosis found in long-term hemodialysis patients. Localized forms of amyloidosis include, without limitation, diabetes type II and any related disorders thereof, neurodegenerative diseases such as scrapie, bovine spongiform encephalitis, Creutzfeldt-Jakob disease, Alzheimer's disease, Cerebral Amyloid Angiopathy, and prion protein related disorders.

Except as otherwise expressly defined herein, the abbreviations used herein for designating the amino acids and the protective groups are based on recommendations of the IUPAC-IUB Commission on Biochemical Nomenclature (Biochemistry, 1972, 11:1726-1732).

The Aβ(16-21) site is known to play an important role in initiating the harmful process of Aβ peptide amyloidogenesis. It is also known that when these peptides are made from D-amino acids, they retain their ability to interact with the natural all-L-homologous sequence, thereby preventing amyloidogenesis.

Other amyloid proteins which may be used in the present invention include, without limitation, IAPP, β2-microglubeline, amyloid A protein, and prion-related proteins.

The vaccine of the present invention, prepared from all-D-Aβ(16-21), D-Aβ(10-16), D-Aβ(1-40), D-Aβ(1-42) or the C-terminal region of D-Aβ(1-42), is believed to elicit an immune response in the host or in producing antibodies that recognize the naturally occurring target. As used herein, “all-D” includes peptides having ≧75%, ≧80%, ≧85%, ≧90%, ≧95%, and 100% D-configuration amino acids. Also, the vaccine of the present invention does not present the drawbacks of using “self” proteins and does not need to be aggregated to induce an immune response. For example, the antibodies raised against the all-D-Aβ(16-21) peptide can be expected to recognize the all-L-Aβ(16-21) peptide sequence.

The elicited antibodies present in the host having received the vaccine of the present invention bind at the Aβ(16-21) site or other sites such as the C-terminal region of Aβ and have the same or even greater ability to prevent amyloidogenesis as do the short peptides themselves. The vaccine of the present invention causes the generation of effective antiamyloidogenic antibodies in the vaccinated host.

A suggested immunization procedure is as follows:
a) prepare a vaccine from an all-D peptide having a sequence substantially the same as that of a naturally occurring β amyloid peptide, namely Aβ (all-L). The all-D peptide includes a full length Aβ (1-42, all-D), a peptide derived from an immunogenic fragment of Aβ (1-42, all-D), and a related peptidomimetic;
b) immunize a host with the vaccine to generate an antibody in the host with a binding site capable of preventing fibrillogenesis.

Suitable pharmaceutically acceptable carriers include, without limitation, any non-immunogenic pharmaceutical adjuvants suitable for oral, parenteral, intravascular (IV), intraarterial (IA), intramuscular (IM), and subcutaneous (SC) administration routes, such as phosphate buffer saline (PBS).

The pharmaceutical carriers may contain a vehicle, which carries antigens to antigen-presenting cells. Examples of vehicles are liposomes, immune-stimulating complexes, microfluidized squalene-in-water emulsions, microspheres which may be composed of poly(lactic/glycolic) acid (PLGA). Particulates of defined dimensions (<5 micron) include, without limitation, oil-in-water microemulsion (MF59) and polymeric microparticules.

The carriers of the present invention may also include chemical and genetic adjuvants to augment immune responses or to increase the antigenicity of antigenic immunogens. These adjuvants exert their immunomodulatory properties through several mechanisms such as lymphoid cells recruitment, cytokine induction, and the facilitation of DNA entry into cells. Cytokine adjuvants include, without limitation, granulocyte-macrophage colony-stimulating factor, interleukin-12, GM-CSF, synthetic muramyl dipeptide analog or monophosphoryl lipid A. Other chemical adjuvants include, without limitation, lactic acid bacteria, Al(OH)₃, muramyl dipeptides and saponins.

The peptide may be coupled to a carrier that will modulate the half-life of the circulating peptide. This will allow the control on the period of protection. The peptide-carrier may also be emulsified in an adjuvant and administrated by usual immunization route.

The vaccine of the present invention will, for the most part, be administered parenterally, such as intravascularly (IV), intraarterially (IA), intramuscularly (IM), subcutaneously (SC), or the like. In some instances, administration may be oral, nasal, rectal, transdermal or aerosol, where the nature of the vaccine allows for transfer to the vascular system. Usually a single injection will be employed although more than one injection may be used, if desired. The vaccine may be administered by any convenient means, including syringe, trocar, catheter, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix.

The use of the vaccine of the present invention in preventing and/or treating Alzheimer's disease and other amyloid related diseases can be validated by raising antibodies against the corresponding all-D peptide and testing them to see if they can effectively inhibit or prevent the fibrillogenesis of the natural amyloid peptide (all-L).

The compounds used to prepare vaccines in accordance with the present invention have the common structure of Formula I:
R∝—(P)—R″ (I),
wherein
P is an all-D peptide interacting with at least one region of an amyloid protein, e.g., β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent selected from the group consisting of:
   hydrogen;
   lower alkyl groups, e.g., acyclic or cyclic having 1 to 8 carbon atoms, without or with functional groups, e.g., carboxylate, sulfonate and phosphonate;
   aromatic groups;
   heterocyclic groups; and
   acyl groups, e.g., alkylcarbonyl, arylcarbonyl, sulfonyl and phosphonyl groups; and
R″ is a C-terminal substituent, e.g., hydroxy, alkoxy, aryloxy, unsubstituted or substituted amino groups.
R′ and R″ may be identical or different; the alkyl or aryl group of R′ and R″ may further be substituted with organic functionalities selected from the group of halides (F, Cl, Br, and I), hydroxyl, alkoxyl, aryloxyl, hydroxycarbonyl, alkoxylcarbonyl, aryloxycarbonyl, unsubstituted or substituted amino, sulfo or alkyloxysulfonyl, phosphono or alkoxyphosphonyl, and the like.

Where a functional group is an acid, its pharmaceutically acceptable salt or ester is in the scope of this invention. Where a functional group is a base, its pharmaceutically acceptable salt is in the scope of this invention.

In one embodiment, the preferred compounds are selected from the full-length peptide, Aβ (1-42, all-D), and its lower homologues consisting of Aβ (1-40, all-D), Aβ (1-35, all-D), and Aβ (1-28, all-D).

In another embodiment, the preferred compounds are selected from a group of short peptides, e.g., Aβ (1-7, all-D), Aβ (10-16, all-D), Aβ (16-21, all-D), Aβ (36-42, all-D). The peptides can be shortened further by removing one or more residues from either end or both ends.

The preferred compounds may also be all-D peptides derived from the peptides above by substitution of one or more residues in the naturally occurring sequence. In another embodiment, the preferred compounds are peptidomimetics of the above-said peptides.

In a further embodiment, the preferred compounds may be coupled with a carrier that will modulate the biodistribution, immunogenic property and the half-life of the compounds.

The following are exemplary compounds for preparing vaccines for preventing or treating Alzheimer's disease and other amyloid related diseases:
1 Aβ (1-42, all-D)
2 Aβ (1-40, all-D)
3 Aβ (1-35, all-D)
4 Aβ (1-28, all-D)
5 Aβ (1-7, all-D)
6 Aβ (10-16, all-D)
7 Aβ (16-21, all-D)
8 Aβ (36-42, all-D)
9 Lys-Ile-Val-Phe-Phe-Ala (all-D)
10 Lys-Lys-Leu-Val-Phe-Phe-Ala (all-D)
11 Lys-Phe-Val-Phe-Phe-Ala (all-D)
12 Ala-Phe-Phe-Val-Leu-Lys (all-D)
13 Lys-Leu-Val-Phe (all-D)
14 Lys-Ala-Val-Phe-Phe-Ala (all-D)
15 Lys-Leu-Val-Phe-Phe (all-D)
16 Lys-Val-Val-Phe-Phe-Ala (all-D)
17 Lys-Ile-Val-Phe-Phe-Ala-NH₂ (all-D)
18 Lys-Leu-Val-Phe-Phe-Ala-NH₂ (all-D)
19 Lys-Phe-Val-Phe-Phe-Ala-NH₂ (all-D)
20 Ala-Phe-Phe-Val-Leu-Lys-NH₂ (all-D)
21 Lys-Leu-Val-Phe-NH₂ (all-D)
22 Lys-Ala-Val-Phe-Phe-Ala-NH₂ (all-D)
23 Lys-Leu-Val-Phe-Phe-NH₂ (all-D)
24 Lys-Val-Val-Phe-Phe-Ala-NH₂ (all-D)
25 Lys-Leu-Val-Phe-Phe-Ala-Gln (all-D)
26 Lys-Leu-Val-Phe-Phe-Ala-Gln-NH₂ (all-D)
27 His-His-Gln-Lys-Leu-Val-Phe-Phe-Ala-Gln (all-D)
28 Asp-Asp-Asp (all-D)
29 Lys-Val-Asp-Asp-Gln-Asp (all-D)
30 His-His-Gln-Lys (all-D)
31 Phe-Phe-NH—CH₂CH₂SO₃H (all-D)
32 Phe-Phe-NH—CH₂CH₂CH₂SO₃H (all-D)
33 Phe-Phe-NH—CH₂CH₂CH₂CH₂SO₃H (all-D)
34 Phe-Tyr-NH—CH₂CH₂SO₃H (all-D)
35 Phe-Tyr-NH—CH₂CH₂CH₂SO₃H (all-D)
36 Phe-Tyr-NH—CH₂CH₂CH₂CH₂SO₃H (all-D)
37 HO₃SCH₂CH₂-Phe-Phe (all-D)
38 HO₃SCH₂CH₂CH₂-Phe-Phe (all-D)
39 HO₃SCH₂CH₂CH₂CH₂-Phe-Phe (all-D)
40 HO₃SCH₂CH₂-Phe-Tyr (all-D)
41 HO₃SCH₂CH₂CH₂-Phe-Tyr (all-D)
42 HO₃SCH₂CH₂CH₂CH₂-Phe-Tyr (all-D)
43 HO₃SCH₂CH₂-Leu-Val-Phe-Phe-Ala (all-D)
44 HO₃SCH₂CH₂CH₂-Leu-Val-Phe-Phe-Ala (all-D)
45 HO₃SCH₂CH₂CH₂CH₂-Leu-Val-Phe-Phe-Ala (all-D)
46 Leu-Val-Phe-Phe-Ala-NH—CH₂CH₂SO₃H (all-D)
47 Leu-Val-Phe-Phe-Ala-NH—CH₂CH₂CH₂SO₃H (all-D)
48 Leu-Val-Phe-Phe-Ala-NH—CH₂CH₂CH₂CH₂SO₃H (all-D).

The compounds listed above may be modified by removing or inserting one or more amino acid residues, or by substituting one or more amino acid residues with other amino acid or non-amino acid fragments.

The following are exemplary compounds derived from compound 18 (all-D KLVFFA-NH₂) by substituting one or two amino acid residue(s) with other amino acids.
49 Lys-Leu-Val-Trp-Phe-Ala-NH₂ (all-D)
50 Lys-Leu-Val-Phe-Trp-Ala-NH₂ (all-D)
51 Lys-Leu-Val-Trp-Trp-Ala-NH₂ (all-D)
52 Lys-Leu-Val-Tyr-Phe-Ala-NH₂ (all-D)
53 Lys-Leu-Val-Phe-Tyr-Ala-NH₂ (all-D)
54 Lys-Leu-Val-Tyr-Tyr-Ala-NH₂ (all-D)
55 Lys-Leu-Val-Thi-Phe-Ala-NH₂ (all-D)
56 Lys-Leu-Val-Phe-Thi-Ala-NH₂ (all-D)
57 Lys-Leu-Val-Thi-Thi-Ala-NH₂ (all-D)
58 Lys-Leu-Val-Cha-Phe-Ala-NH₂ (all-D)
59 Lys-Leu-Val-Phe-Cha-Ala-NH₂ (all-D)
60 Lys-Leu-Val-Cha-Cha-Ala-NH₂ (all-D)
61 Lys-Leu-Val-Pgly-Phe-Ala-NH₂ (all-D)
62 Lys-Leu-Val-Phe-Pgly-Ala-NH₂ (all-D)
63 Lys-Leu-Val-Pgly-Pgly-Ala-NH₂ (all-D).

For the above compounds, the terms Thi, Cha and Pgly are intended to mean theinylalanine, cyclohexylalanine and phenylglycine, respectively.

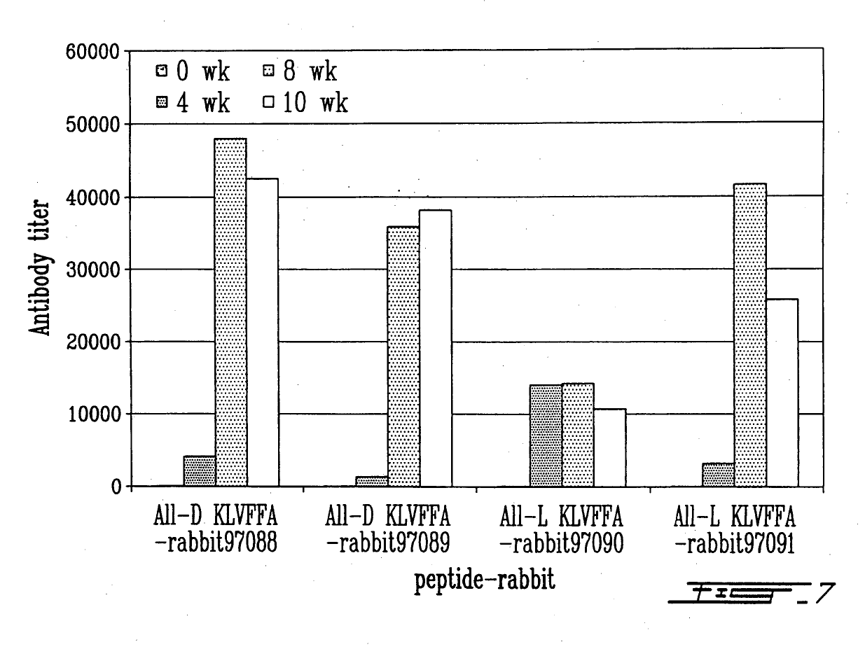

Rabbits were immunized with all-D or all-L KLVFFA. Results of the antibody titers obtained are shown in FIG. 7. As seen in FIG. 7, the vaccine of the present invention causes production of antibodies.

The present invention encompasses various types of immune responses triggered using the vaccine of the present invention, e.g., amyloid therapies using the vaccine approach.

In accordance with the present invention, there is also provided a vaccine which triggers a preferential TH-2 response or a TH-1 response, according to the type of immunization used. By inducing a TH-2 response, anti-inflammatory cytokine production such as IL-4, Il-10 and TGF-β, as well as the production of IgG 1 and IgG 2b antibody classes, are favored. Such type of response would be preferred, as a major inflammatory response in the brain of the patients with AD would be avoided. On the other hand, with a preferred TH-1 response, a pro-inflammatory response with a production of inflammatory cytokines such as IL-1, Il-6, TNF and IFN gamma would be favored. This type of response would more likely trigger activation of the macrophage population. These macrophages would then phagocytose any particulate deposits (such as plaques) via a complement-activated process as well as via antibody-mediated process. This approach would be beneficial to clear already organized senile plaques and prevent the formation of new fibrillary deposits.

Both approaches (i.e. TH-1 and TH-2) are of value. The antigen used could be the peptides which contain regions responsible for cellular adherence, i.e., region 10-16, regions responsible for the GAG binding site, i.e., 13-16, regions responsible for the β sheet 16-21 or regions for 40-42. These peptides could be presented in such a way that either a preferential TH-1 or TH-2 response is obtained, depending on the type of adjuvant used, or depending on the route of administration of the vaccine. For example, a mucosal immunization via nasal administration is possible, since it is known that such a route of administration would favor a TH-2 response.

The present invention will be more readily understood by referring to the following examples, which are given to illustrate the invention rather than to limit its scope.

### EXAMPLE I

An in vitro validation procedure to test the effectiveness of all-D peptide vaccines derived from fibrillogenic proteins was performed in rabbits or mice to demonstrate that antibodies can be raised against AP 16-21 (all-D) (see FIG. 7). The antibodies produced were tested to prove that they effectively prevent the fibrillogenesis of natural AP(1-40, all-L) in vitro. Standard assays for fibrillogenesis were used to evaluate activity, such as those based on Thioflavine T, circular dichroism and solubility.

This approach could also be used to establish which areas of the AP peptide are most effective when used in the form of all-D peptides to prepare antifibrillogenic vaccines. One way this could be performed is as follows:
a) rabbits or mice are immunized with a series of overlapping all-D peptides generated from the Aβ(1-42) sequence, e.g., Aβ(1-6), Aβ(2-8), Aβ(4-10), etc.
b) antisera are prepared from the immunized rabbits or mice.
c) these antisera are tested to see which parts of the Aβ sequence produce antisera which most effectively prevents fibrillogenesis in the standard assays for fibrillogenesis mentioned above.

### EXAMPLE II

Effect of Antibodies Against D- and L-Aβ(16-21) Peptide Vaccine on Fibrillogenesis A validation procedure to test anti-fibrillogenic activity of antibodies raised against D- and L-Aβ(16-21) peptide was performed.

Rabbits were immunized with D- or L-Aβ(16-21) peptide. Antibodies raised were tested for their antifibrillogenic activities by ThT assay and by electron microscopy (EM).

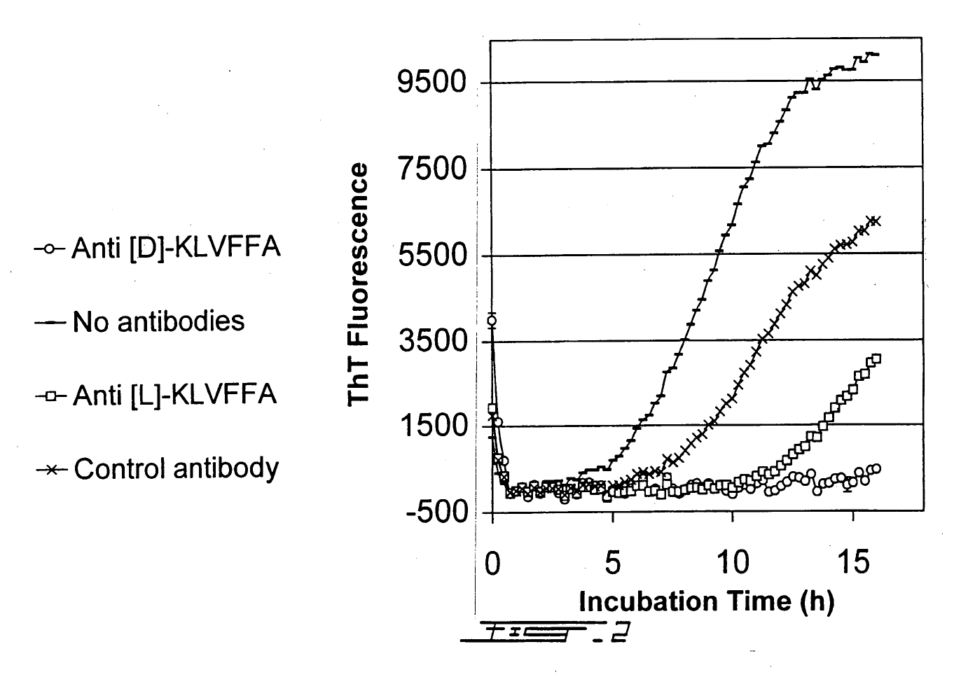

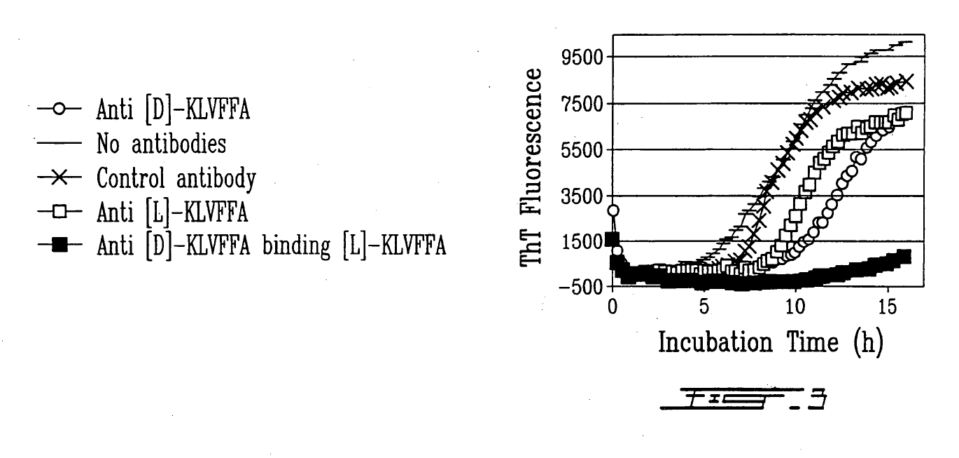

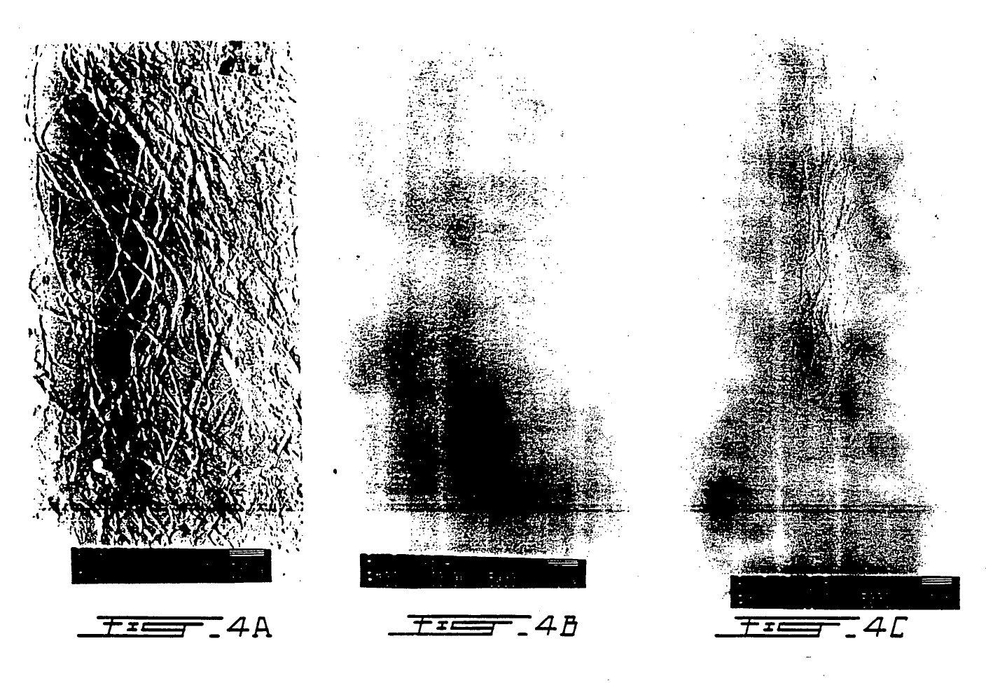

Antibodies raised against the D- and L-forms of KLVFFA were capable of blocking the fibrillogenesis process as seen either by the Thioflavin T assay (ThT) (FIGS. 2 and 3) and by EM (FIGS. 4A to 4C). In the ThT assay, fibril formation is monitored by the increase in fluorescence with time. As seen in the Figures, the antibodies were capable of inhibiting such an increase in fluorescence, proving that these antibodies were inhibiting fibrillogenesis.

As can be seen in these figures (FIGS. 2 to 4), antibodies raised against the D-peptide have a better anti-fibrillogenic activity than anti-L antibodies.

These results were also confirmed by EM (FIGS. 4A to 4C) where both anti-D and anti-L KLVFFA peptide blocked the fibril formation when compared to control (FIG. 4A). Moreover, again the anti-D peptide has a greater anti-fibrillogenic activity (FIG. 4B) than the anti-L peptide (FIG. 4C). This goes along with the ThT assay where the decrease in fluorescence was greater with the anti-D peptide antibody than with the anti-L peptide antibody.

### EXAMPLE III

### Antibody Binding Assay

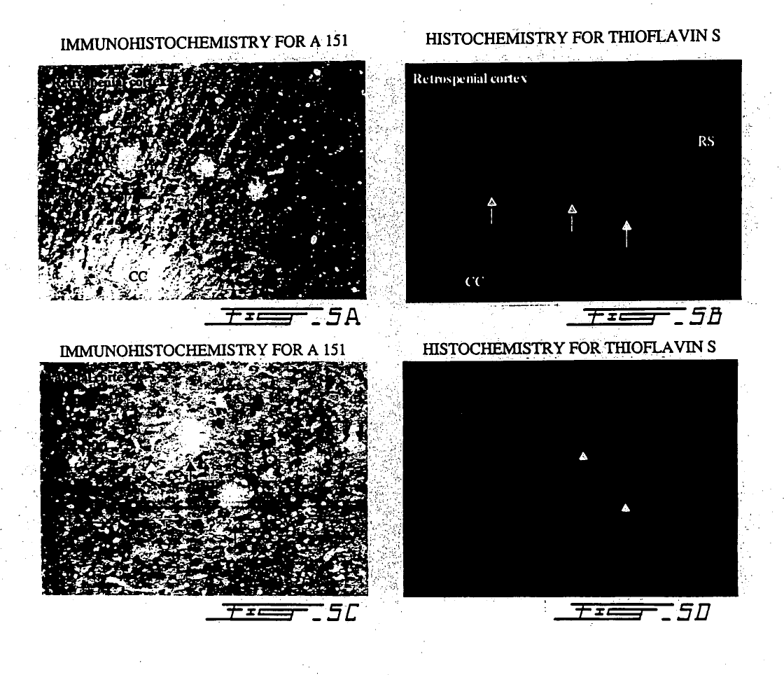

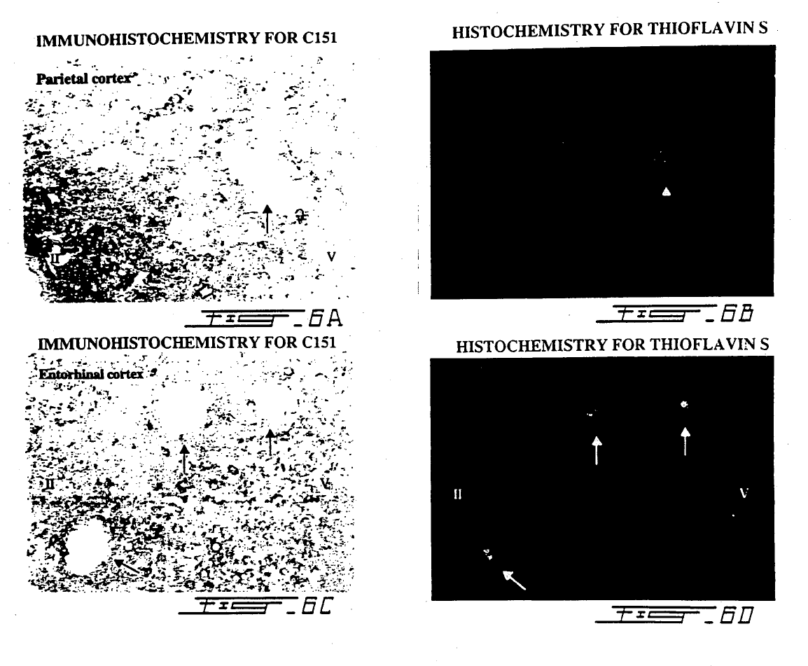

Brain sections were stained with antibodies raised against KLVFFA peptide (D and L forms). As seen in FIGS. 5A to 5D and 6A to 6D, the antibodies were not capable of binding to aggregated (ThioS positive) Ap. It can be seen from both sets of figures, which were stained for both plaques (ThioS) and anti-peptides that the antibodies are recognizing Aβ at the surface of the cells but are not capable of binding to plaques. These results show that the anti-KLVFFA peptide antibody is recognizing the non-fibrillary Aβ but does not bind to aggregated Aβ. There was no difference between the anti-D and anti-L peptide antibodies in this assay.

These results clearly prove that the antibody recognizes only the non-aggregated form and blocks the fibrillogenesis. By having such activity, the vaccine of the present invention 1) prevents Aβ from organizing itself into a fibril and 2) prevents an inflammatory response being triggered by such an antibody binding to an insoluble form, since the antibody is not able to bind to aggregated Aβ.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for preventing and/or treating an amyloid-related disease in a subject, comprising administering to a subject an antigenic amount of an all-D peptide which elicits production of antibodies against said all-D peptide, and elicit an immune response by said subject, therefore preventing fibrillogenesis and associated cellular toxicity, wherein said antibodies and/or immune cells interact with at least one region of an amyloid protein selected from the group consisting of β sheet region and GAG-binding site region, immunogenic fragments thereof, protein conjugates thereof, immunogenic derivative peptides thereof, immunogenic peptides thereof, and immunogenic peptidomimetics thereof,

2. The method of claim 0, wherein said compound is a compound of Formula I:
R′—(P)—R″ (I),
wherein
P is an all-D peptide interacting with at least one region of an amyloid protein selected from the group consisting of β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent selected from the group consisting of:
hydrogen;
lower alkyl groups selected from the group consisting of acyclic or cyclic having 1 to 8 carbon atoms;
aromatic groups;
heterocyclic groups; and
acyl groups; and
R″ is a C-terminal substituent selected from the group consisting of hydroxy, alkoxy, aryloxy, unsubstituted or substituted amino groups.

3. The method of claim 0, wherein said alkyl or aryl group of R′ and R″ is further substituted with halide; hydroxyl, alkoxyl, aryloxyl, hydroxycarbonyl, alkoxylcarbonyl, aryloxycarbonyl, carbamyl, unsubstituted or substituted amino, sulfo or alkyloxysulfonyl, phosphono or alkoxyphosphonyl groups.

4. The method of claim 0, wherein said compound further comprises an acid functional group, a pharmaceutically acceptable salt or ester form thereof; or a base functional group or pharmaceutically acceptable salt form thereof.

5. The method of claim 0, wherein said compound is selected from the group consisting of compounds 1 to 48.

6. The method of claim 0, wherein said compound is modified by removing or inserting one or more amino acid residues, or by substituting one or more amino acid residues with other amino acid or non-amino acid fragment.

7. The method of claim 0, wherein said compound is selected from the group consisting of compounds 49 to 63.

8. The method of claim 0, wherein said subject is a human being.

9. The method of claim 0, wherein said disease is Alzheimer's disease.

10. A vaccine for preventing and/or treating an amyloid-related disease in a subject, comprising an antibody raised against an antigenic amount of an all-D peptide which interacts with at least one region of an amyloid protein selected from the group consisting of β sheet region and GAG-binding site region, Aβ (1-42, all-D), immunogenic fragments thereof, protein conjugates thereof, immunogenic derivative peptides thereof, immunogenic peptides thereof, and immunogenic peptidomimetics thereof, wherein said antibody interacts with amyloid proteins and therefore prevents fibrillogenesis.

11. The vaccine of claim 0, wherein said compound is a compound of Formula I:
R′—(P)—R″ (I),
wherein
P is an all-D peptide interacting with at least one region of an amyloid protein selected from the group consisting of β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent selected from the group consisting of:
hydrogen;
lower alkyl groups selected from the group consisting of acyclic or cyclic having 1 to 8 carbon atoms;
aromatic groups;
heterocyclic groups; and
acyl groups; and
R″ is a C-terminal substituent selected from the group consisting of hydroxy, alkoxy, aryloxy, unsubstituted or substituted amino groups.

12. The vaccine of claim 0, wherein said alkyl or aryl group of R′ and R″ are further substituted with halide; hydroxyl, alkoxyl, aryloxyl, hydroxycarbonyl, alkoxylcarbonyl, aryloxycarbonyl, carbamyl, unsubstituted or substituted amino, sulfo or alkyloxysulfonyl, phosphono or alkoxyphosphonyl groups.

13. The vaccine of claim 0, wherein said compound further comprises an acid functional group, a pharmaceutically acceptable salt or ester form thereof; or a base functional group or pharmaceutically acceptable salt form thereof.

14. The vaccine of claim 0, wherein said compound is selected from the group consisting of compounds 1 to 48.

15. The vaccine of claim 0, wherein said compound is modified by removing or inserting one or more amino acid residues, or by substituting one or more amino acid residues with other amino acid or non-amino acid fragment.

16. The method of claim 0, wherein said compound is selected from the group consisting of compounds 49 to 63.

17. The vaccine of claim 0, wherein said subject is a human being.

18. The vaccine of claim 0, wherein said disease is Alzheimer's disease.

19. A method for preventing and/or treating an amyloid-related disease in a subject, comprising administering to said subject an antigenic amount of an all-D peptide which interacts with at least one region of an amyloid protein selected from the group consisting of β sheet region and GAG-binding site region, immunogenic fragments thereof, protein conjugates thereof, immunogenic derivative peptides thereof, immunogenic peptides thereof, and immunogenic peptidomimetics thereof, wherein said compound elicits an immune response by said subject and therefore prevents fibrillogenesis.

20. The method of claim 0, wherein said compound is a compound of Formula I:
R′—(P)—R″ (I),
wherein
P is an all-D peptide interacting with at least one region of an amyloid protein selected from the group consisting of β sheet region and GAG-binding site region, Aβ (1-42, all-D), immunogenic fragments thereof, immunogenic derivatives thereof, protein conjugates thereof, immunogenic peptides thereof, and immunogenic peptidomimetics thereof;
R′ is an N-terminal substituent selected from the group consisting of:
hydrogen;
lower alkyl groups selected from the group consisting of acyclic or cyclic having 1 to 8 carbon atoms;
aromatic groups;
heterocyclic groups; and
acyl groups; and
R″ is a C-terminal substituent selected from the group consisting of hydroxy, alkoxy, aryloxy, unsubstituted or substituted amino groups.

21-45. (canceled).


SEQUENCE LISTING (63 sequences)

SEQ ID NO: 1 - protein, 42 aa, Artificial Sequence [sequence omitted]

SEQ ID NO: 2 - protein, 40 aa, Artificial Sequence
  Description of Artificial Sequence All D peptides or peptidomimetics
DAEFRHDSGYEVHHQKLVFFAEDVGSNKGAIIGLMVGGVV

SEQ ID NO: 3 - protein, 35 aa, Artificial Sequence
  Description of Artificial Sequence All D peptides or peptidomimetics
DAEFRHDSGYEVHHQKLVFFAEDVGSNKGAIIGLM

SEQ ID NO: 4 - protein, 28 aa, Artificial Sequence
  Description of Artificial Sequence All D peptides or peptidomimetics
DAEFRHDSGYEVHHQKLVFFAEDVGSNK

SEQ ID NO: 5 - protein, 7 aa, Artificial Sequence
  Description of Artificial Sequence All D peptides or peptidomimetics
DAEFRHD

SEQ ID NO: 6 - protein, 7 aa, Artificial Sequence
  Description of Artificial Sequence All D peptides or peptidomimetics
YEVHHQK

SEQ ID NO: 7 - protein, 6 aa, Artificial Sequence
  Description of Artificial Sequence All D peptides or peptidomimetics
KLVFFA

SEQ ID NO: 8 - protein, 7 aa, Artificial Sequence
  Description of Artificial Sequence All D peptides or peptidomimetics
VGGVVIA

SEQ ID NO: 9 - protein, 6 aa, Artificial Sequence
  Description of Artificial Sequence All D peptides or peptidomimetics
KIVFFA

SEQ ID NO: 10 - protein, 7 aa, Artificial Sequence
  Description of Artificial Sequence All D peptides or peptidomimetics
KKLVFFA

SEQ ID NO: 11 - protein, 6 aa, Artificial Sequence
  Description of Artificial Sequence All D peptides or peptidomimetics
KFVFFA

SEQ ID NO: 12 - protein, 6 aa, Artificial Sequence
  Description of Artificial Sequence All D peptides or peptidomimetics
AFFVLK

SEQ ID NO: 13 - protein, 4 aa, Artificial Sequence
  Description of Artificial Sequence All D peptides or peptidomimetics
KLVF

SEQ ID NO: 14 - protein, 6 aa, Artificial Sequence
  Description of Artificial Sequence All D peptides or peptidomimetics
KAVFFA

SEQ ID NO: 15 - protein, 5 aa, Artificial Sequence
  Description of Artificial Sequence All D peptides or peptidomimetics
KLVFF

SEQ ID NO: 16 - protein, 6 aa, Artificial Sequence
  Description of Artificial Sequence All D peptides or peptidomimetics
KVVFFA

SEQ ID NO: 17 - protein, 6 aa, Artificial Sequence
  Description of Artificial Sequence All D peptides or peptidomimetics
KIVFFA

SEQ ID NO: 18 - protein, 6 aa, Artificial Sequence
  Description of Artificial Sequence All D peptides or peptidomimetics
KLVFFA

SEQ ID NO: 19 - protein, 6 aa, Artificial Sequence
  Description of Artificial Sequence All D peptides or peptidomimetics
KFVFFA

SEQ ID NO: 20 - protein, 6 aa, Artificial Sequence
  Description of Artificial Sequence All D peptides or peptidomimetics
AFFVLK

SEQ ID NO: 21 - protein, 4 aa, Artificial Sequence
  Description of Artificial Sequence All D peptides or peptidomimetics
KLVF

SEQ ID NO: 22 - protein, 6 aa, Artificial Sequence
  Description of Artificial Sequence All D peptides or peptidomimetics
KAVFFA

SEQ ID NO: 23 - protein, 5 aa, Artificial Sequence
  Description of Artificial Sequence All D peptides or peptidomimetics
KLVFF

SEQ ID NO: 24 - protein, 6 aa, Artificial Sequence
  Description of Artificial Sequence All D peptides or peptidomimetics
KVVFFA

SEQ ID NO: 25 - protein, 7 aa, Artificial Sequence
  Description of Artificial Sequence All D peptides or peptidomimetics
KLVFFAE

SEQ ID NO: 26 - protein, 7 aa, Artificial Sequence
  Description of Artificial Sequence All D peptides or peptidomimetics
KLVFFAE

SEQ ID NO: 27 - protein, 10 aa, Artificial Sequence
  Description of Artificial Sequence All D peptides or peptidomimetics
HHQKLVFFAE

SEQ ID NO: 28 - protein, 3 aa, Artificial Sequence
  Description of Artificial Sequence All D peptides or peptidomimetics
DDD

SEQ ID NO: 29 - protein, 6 aa, Artificial Sequence
  Description of Artificial Sequence All D peptides or peptidomimetics
KVDDQD

SEQ ID NO: 30 - protein, 4 aa, Artificial Sequence
  Description of Artificial Sequence All D peptides or peptidomimetics
HHQK

SEQ ID NO: 31 - protein, 2 aa, Artificial Sequence
  Description of Artificial Sequence All D peptides or peptidomimetics
FF

SEQ ID NO: 32 - protein, 2 aa, Artificial Sequence
  Description of Artificial Sequence All D peptides or peptidomimetics
FF

SEQ ID NO: 33 - protein, 2 aa, Artificial Sequence
  Description of Artificial Sequence All D peptides or peptidomimetics
FF

SEQ ID NO: 34 - protein, 2 aa, Artificial Sequence
  Description of Artificial Sequence All D peptides or peptidomimetics
FY

SEQ ID NO: 35 - protein, 2 aa, Artificial Sequence
  Description of Artificial Sequence All D peptides or peptidomimetics
FY

SEQ ID NO: 36 - protein, 2 aa, Artificial Sequence
  Description of Artificial Sequence All D peptides or peptidomimetics
FY

SEQ ID NO: 37 - protein, 2 aa, Artificial Sequence
  Description of Artificial Sequence All D peptides or peptidomimetics
FF

SEQ ID NO: 38 - protein, 2 aa, Artificial Sequence
  Description of Artificial Sequence All D peptides or peptidomimetics
FF

SEQ ID NO: 39 - protein, 2 aa, Artificial Sequence
  Description of Artificial Sequence All D peptides or peptidomimetics
FF

SEQ ID NO: 40 - protein, 2 aa, Artificial Sequence
  Description of Artificial Sequence All D peptides or peptidomimetics
FY

SEQ ID NO: 41 - protein, 2 aa, Artificial Sequence
  Description of Artificial Sequence All D peptides or peptidomimetics
FY

SEQ ID NO: 42 - protein, 2 aa, Artificial Sequence
  Description of Artificial Sequence All D peptides or peptidomimetics
FY

SEQ ID NO: 43 - protein, 5 aa, Artificial Sequence
  Description of Artificial Sequence All D peptides or peptidomimetics
LVFFA

SEQ ID NO: 44 - protein, 5 aa, Artificial Sequence
  Description of Artificial Sequence All D peptides or peptidomimetics
LVFFA

SEQ ID NO: 45 - protein, 5 aa, Artificial Sequence
  Description of Artificial Sequence All D peptides or peptidomimetics
LVFFA

SEQ ID NO: 46 - protein, 5 aa, Artificial Sequence
  Description of Artificial Sequence All D peptides or peptidomimetics
LVFFA

SEQ ID NO: 47 - protein, 5 aa, Artificial Sequence
  Description of Artificial Sequence All D peptides or peptidomimetics
LVFFA

SEQ ID NO: 48 - protein, 5 aa, Artificial Sequence
  Description of Artificial Sequence All D peptides or peptidomimetics
LVFFA

SEQ ID NO: 49 - protein, 6 aa, Artificial Sequence
  Description of Artificial Sequence All D peptides or peptidomimetics
KLVWFA

SEQ ID NO: 50 - protein, 6 aa, Artificial Sequence
  Description of Artificial Sequence All D peptides or peptidomimetics
KLVFWA

SEQ ID NO: 51 - protein, 6 aa, Artificial Sequence
  Description of Artificial Sequence All D peptides or peptidomimetics
KLVWWA

SEQ ID NO: 52 - protein, 6 aa, Artificial Sequence
  Description of Artificial Sequence All D peptides or peptidomimetics
KLVYFA

SEQ ID NO: 53 - protein, 6 aa, Artificial Sequence
  Description of Artificial Sequence All D peptides or peptidomimetics
KLVFYA

SEQ ID NO: 54 - protein, 6 aa, Artificial Sequence
  Description of Artificial Sequence All D peptides or peptidomimetics
KLVYYA

SEQ ID NO: 55 - protein, 6 aa, Artificial Sequence
  Description of Artificial Sequence All D peptides or peptidomimetics
KLVXFA

SEQ ID NO: 56 - protein, 6 aa, Artificial Sequence
  Description of Artificial Sequence All D peptides or peptidomimetics
KLVFXA

SEQ ID NO: 57 - protein, 6 aa, Artificial Sequence
  Description of Artificial Sequence All D peptides or peptidomimetics
KLVXXA

SEQ ID NO: 58 - protein, 6 aa, Artificial Sequence
  Description of Artificial Sequence All D peptides or peptidomimetics
KLVXFA

SEQ ID NO: 59 - protein, 6 aa, Artificial Sequence
  Description of Artificial Sequence All D peptides or peptidomimetics
KLVFXA

SEQ ID NO: 60 - protein, 6 aa, Artificial Sequence
  Description of Artificial Sequence All D peptides or peptidomimetics
KLVXXA

SEQ ID NO: 61 - protein, 6 aa, Artificial Sequence
  Description of Artificial Sequence All D peptides or peptidomimetics
KLVXFA

SEQ ID NO: 62 - protein, 6 aa, Artificial Sequence
  Description of Artificial Sequence All D peptides or peptidomimetics
KLVFXA

SEQ ID NO: 63 - protein, 6 aa, Artificial Sequence
  Description of Artificial Sequence All D peptides or peptidomimetics
KLVXXA